(19) United States
(12) Patent Application Publication
(10) Pub. No.: US 2020/0345403 A1
(43) Pub. Date: Nov. 5, 2020

(54) INSTRUMENTS AND METHODOLOGY INVOLVING CRYOABLATION

(71) Applicant: The Board of Trustees of the Leland Stanford Junior University, Stanford, CA (US)

(72) Inventors: Paul J. Wang, Saratoga, CA (US); Neal K. Bhatia, Stanford, CA (US); Meghedi Babakhanian, Stanford, CA (US)

(21) Appl. No.: 16/866,388

(22) Filed: May 4, 2020

Related U.S. Application Data
(60) Provisional application No. 62/843,117, filed on May 3, 2019.

(51) Int. Cl. A61B 18/02

(52) U.S. Cl. CPC A61B 18/02; A61B 2018/00577

(57) ABSTRACT

In specific examples, aspects are directed towards an inner lumen which is at least partially moveable and/or slidable relative an outer lumen. The inner lumen is allowed to extend outside the outer lumen while the outer lumen remains stationary in a relative position to or outside the target tissue site. The inner lumen may then use a gas or liquid to cause a decrease in temperature as it expands with a sudden drop in pressure. This cools the target tissue sufficiently to cryoablate portions of the tissue at various depth.

### OVERVIEW

Aspects of various embodiments are directed to instruments and methods involving cryoablation procedures.

One example non-limiting application of cryoablation involves Ventricular Tachycardia (VT) which may continue to recur despite advances in cardiac ablation technologies and methodologies. This is often due to an inability to reach in an effective manner intramural focus, which may create transmural lesions, and inability to eliminate abnormal ventricular substrate. Ineffective treatment in these regards may result in recurrence of VT and/or other types of arrhythmia. As applied in other applications, ineffective or incomplete treatment of cryoablation may result in recurrence of a variety of conditions including nerve/neurologic conditions among other symptom-specific anomalies.

Various example embodiments are directed to issues such as those addressed above and/or others which may become apparent from the following disclosure involving cryoablation of tissue at various tissue depths.

In one example, aspects of the present disclosure are directed to an instrument having an inner lumen and outer lumen arranged for use in a cryoenergy needle-like apparatus for improved delivery of cryoenergy where depth into the tissue may be important and/or for improved transmural lesions by using a cryoablation method for cardiac ablation.

In more particular examples, aspects of the present disclosure are directed to use of an inner lumen which is at least partially moveable and/or slidable within an outer lumen. The inner lumen may have apertures along a length dimension aligned with a depth dimension of the target cardiac tissue and when the inner lumen is moved, a liquid or gas results in cryoenergy being applied to the tissue via the aligned apertures.

In another example, aspects of the present disclosure are directed to an inner lumen which is at least partially moveable and/or slidable, along its length, relative an outer lumen. The outer lumen has an outer portion that facilitates maintaining a stationary position relative to the target tissue. Movement of the inner lumen may then cause a gas or liquid to be applied at various depth levels within the target tissue.

In more particular example, aspects of the present disclosure are directed to an inner lumen which is at least partially moveable and/or slidable relative an outer lumen. The inner lumen is allowed to extend outside the outer lumen while the outer lumen remains stationary in a relative position to or outside the target tissue site. The inner lumen may then use a gas or liquid to cause a decrease in temperature as it expands with a sudden drop in pressure. This cools the target tissue sufficiently to cryoablate portions of the tissue at various depth.

In another particular example, aspects of the present disclosure are directed to an inner lumen which is at least partially moveable and/or slidable relative an outer lumen. The inner lumen is allowed to slide within the outer lumen while the outer lumen remains stationary within the target tissue.

In yet another particular example, an outer lumen permits an inner lumen to move relative to the outer lumen. This allows the outer lumen to maintain a stationary position relative to a target tissue site while the inner lumen moves within the outer lumen.

In more specific examples, a plunger may be controlled manually and/or via robot used to effect step-wise control of the movements of the inner lumen.

Another example is directed to a method involving the use of an inner lumen within an outer lumen. The method permits movement of the inner lumen relative to the outer lumen, and while the outer lumen maintains a stationary position relative to a target tissue site, the inner lumen moves within the outer lumen with the inner lumen at least partially within the outer lumen. While a plurality of delivery positions along a length dimension of the inner lumen are aligned with a depth dimension of tissue in the target tissue site, using a temperature of gas or liquid at the plurality of delivery positions in the inner lumen to effect cryoablation of portions of the tissue at a plurality of depths within tissue. The inner lumen and the outer lumen are cooperatively configured so that movement of the inner lumen causes the temperature of the liquid or gas to be applied at different depth levels of the target tissue site via different ones of the plurality of delivery positions

The above discussion is not intended to describe each embodiment or every implementation of the present disclosure.

### BRIEF DESCRIPTION OF THE DRAWING

Various example embodiments may be more completely understood in consideration of the following detailed description in connection with the accompanying drawings, in which:

FIGS. 1A, 1B and 1C respectively illustrate different types of apparatuses or instruments at different showing different states of use or application in accordance with examples consistent with aspects of the present disclosure;

FIG. 2A depicts an apparatus or instrument with a solid outer lumen and a center perforated inner lumen in a partially-retracted position, in accordance with examples of the present disclosure;

FIG. 2B depicts an apparatus or instrument with a solid outer lumen and a side-perforated/tapered inner lumen with a blocker, and shown in a partially retracted position, in accordance with examples of the present disclosure;

FIG. 3A depicts an apparatus or instrument with a solid outer lumen and a spirally perforated inner lumen in a fully extended position, in accordance examples of the present disclosure;

FIG. 3B depicts an apparatus or instrument with a solid outer lumen and a spirally perforated inner lumen in a fully extended position, and including an optional pointed tip, in accordance with examples of the present disclosure;

FIG. 4A depicts an interior, modified cross-sectional view of an apparatus or instrument with an outer lumen and an inner lumen along with a sheath/cover over the inner lumen, in accordance with examples of the present disclosure; and

FIG. 4B depicts a view of the apparatus or instrument of FIG. 4A with an outer lumen and an inner lumen along with the sheath/cover align with delivery positions of the inner lumen, in accordance with examples of the present disclosure.

While various embodiments discussed herein are amenable to modifications and alternative forms, aspects thereof have been shown by way of example in the drawings and will be described in detail. It should be understood, however, that the intention is not to limit the disclosure to the particular embodiments described. In addition, the term “example” as used throughout this application is only by way of illustration, and not limitation.

### FURTHER DISCUSSION

Aspects of the present disclosure are believed to be applicable to a variety of procedures involving and benefitting cryoablation patients. In certain specific embodiments, aspects of the present disclosure are exemplified in connection with instruments and methods involving an outer lumen and a cooperatively configured inner lumen which may be introduced via a catheter, to cardiac tissue at multiple tissue depths corresponding to positions of a moveable inner lumen for treating, or freezing/cryoablating, such tissue. While the present disclosure is not necessarily limited to such aspects, an understanding of specific examples in the following description may be understood from discussion in such specific contexts.

Accordingly, in the following description various specific details are set forth to describe specific examples presented herein. It should be apparent to one skilled in the art, however, that one or more other examples and/or variations of these examples may be practiced without all the specific details given below. In other instances, well known features have not been described in detail so as not to obscure the description of the examples herein. For ease of illustration, the same reference numerals may be used in different diagrams to refer to the same elements or additional instances of the same element. Also, although aspects and features may in some cases be described in individual figures, it will be appreciated that features from one figure or embodiment can be combined with features of another figure or embodiment even though the combination is not explicitly shown or explicitly described as a combination.

In specific examples, aspects of the present disclosure are directed to an inner lumen which is at least partially moveable and/or slidable, along its length, relative an outer lumen. The outer lumen has an outer portion, such as a surface portion, that may be used or configured to maintain the outer lumen at a stationary position relative to the target tissue in order to maintain the stationary position while the inner lumen moves for presentation of a gas or liquid to be applied at various depth levels within the tissue, with the depth levels associated with different delivery positions of the inner lumen.

In other specific examples consistent with the above-described aspects, an outer lumen has a length longer than an inner lumen to allow the inner lumen to move and/or slide within the outer lumen. In this example the outer lumen may be inserted into the tissue containing or corresponding to the target area. For example, while the outer lumen remains at a fixed position relative to target area of the tissue, the inner lumen may be slid within the outer lumen to position the tip of the inner lumen, which depending on the design may be the coldest portion of the liquid or gas, nearest a selected depth into the targeted tissue.

In yet another specific example, the outer lumen may be open at the distal end and allowing for the inner lumen to be extended outside of the outer lumen. The inner lumen may be fabricated with an optionally tapered/pointed distal end to ease entry into the surrounding tissue.

In more specific examples, the inner lumen may be constructed with delivery positions implemented via apertures or tapered thickness portions to allow for the gas or liquid to flow in the inner lumen to escape, thereby causing such portion(s) of the lumen be the coldest for delivery to the target tissue; as examples, with the delivery positions of the inner lumen moved within the outer lumen towards, from and/or through the distal end of the outer lumen. Examples of the delivery positions of the inner lumen may include, but are not limited to: a singular aperture near at the distal end of the inner lumen; a plurality of apertures perforating the sides of the inner lumen; and sets of apertures arranged linearly and/or nonlinearly along the length of the inner lumen such as in an array and/or a spiral pattern so to effect delivery of the cryoenergy at different magnitudes and, due to the tissue and/or chemistry affecting the temperature of the deliverable cryoenergy, at different magnitudes for different delivery timings.

According to additional aspects of the disclosure which may be combined with one or more of the above aspects, a cryoenergy-delivery instrument or assembly may also include a sheath positioned between in the inner and outer lumens. The sheath may be movable relative to the inner lumen so as to allow it to cover at least a portion of the inner lumen. In more specific examples involving such sheath-specific embodiments: the sheath may cover the entirety of the delivery positions of the inner lumen and then moved relative to the inner lumen (e.g., sheath being pulled back away from the distal end of the outer lumen once the inner lumen is positioned); the sheath may cover a subset of the delivery positions except for the most distal position of the inner lumen (e.g., to maintain the gas or liquid in a condition or at or below a threshold temperature); the sheath and the inner lumen may be arranged to permit the sheath to cover the inner lumen until a distal/delivery portion of the inner lumen (e.g., once the outer lumen is secured relative to or at the epicardium) is moved through the end of the outer lumen for delivery of the cryoenergy via the tip and/or via the delivery positions along the length of the inner lumen; and the sheath may positioned to be fixed within and/or throughout the length of outer lumen as the inner lumen moves relative to the outer lumen to effect delivery of the cryoenergy.

Further, in various ones of these sheath-specific examples, the sheath may be at least partially, if not entirely rigid along the length of the inner lumen, and/or the sheath may be gatherable at least near the distal (delivery) end or portion of the inner lumen so as to provide a gathered sheath end-portion which maintains the end portion of the inner lumen at a certain state, position and/or temperature in preparation for such delivery of cryoenergy and/or for actually applying such cryoenergy.

According to another aspect of the disclosure, an assembly may also include reefing material which can be me moved relative to the inner lumen by gathering to expose more or less of the inner lumen.

In yet other specific examples consistent with the present disclosure, the inner lumen may have a blocker at the distal end of the assembly and may be used to apply and/or block the flow of liquid or gas from traveling out of the inner lumen into the distal end of the outer lumen. Also, the length of the penetrating portion of such a needle-like instrument may be constructed to depend on the thickness of the tissue being ablated; for example, such designs may include the inner/outer lumen being of a rigid and/or partially-flexible material. In yet further examples, the size of the lesions may be increased by using such a needle-like instrument constructed with small holes to deliver normal saline into the surrounding tissue. Freezing in the manner described above is delivered from a distal to a proximal location.

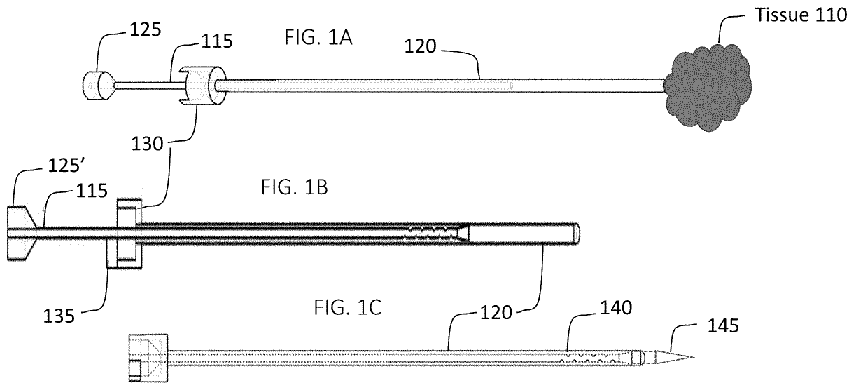

Using cardiac ablation (e.g., for treating VT) as a specific example of use of such above-discussed cryoablation instrument, targeted tissue may treated at certain specific depths via an instrument which may be exemplified by reference to FIGS. 1A, 1B and 1C. Delivery of the cryoenergy to the tissue 110 (FIG. 1A) is achieved by passage of cold liquid delivered through an inner lumen 115 (FIG. 1A) and escaping into an outer lumen 120, which may be through the distal end of the outer lumen 120 into the tissue 110 and/or via the sides of the outer lumen 120. The lumens (as well as sheaths used therewith) may be made of one or more metal(s), plastic(s) or combinations thereof Alternatively, introduction of gas or liquid may occur via an inner lumen 115 and when a pressure change occurs via the Joule-Thomson effect, freezing occurs or when a phase change occurs. In either case the gas or liquid is evacuated from the inner lumen 115 via the outer lumen 120.

In connection with aspects which may be used as part of the instruments illustrated by FIGS. 1A, 1B and 1C, an adapter 125 (FIG. 1A) or 125′ (FIG. 1B) may be used as part of a plunger with manual or automated control for causing the inner lumen 115 to move or slide within the outer lumen 120 towards a receptacle or stop 130 at which stage an optional container or blocker 135 may be engaged to applying block such as the gas or liquid to effect the ablation.

The application may involve, for example, needle cryoablation to achieve ablation of tissue, for example, myocardial tissue, at one or more depths. Advantageously, the above-discussed types of instruments, as illustrated, facilitate delivery of cryoablation energy/freezing throughout the thickness of the tissue, not just at the deepest point the distal end of the outer/inner lumen reaches. Accordingly, the instrument uses a plurality of delivery positions along a length dimension of the inner lumen aligned with a depth dimension of the tissue, and cryoablation is therefore performed at each of these delivery positions spanning from the inner surface of the heart, the endocardium, to the outer surface of the heart, the epicardium; thereby realizing the ability to move the delivery of the cryoablation to the plurality of positions and to freeze throughout the depths, to create a full-thickness transmural lesion.

Another aspect of such above-discussed examples in such a cardiac ablation application is that the endocardial surface presents a warming heat source, making it more difficult to freeze the myocardial tissue that is closest to the endocardial surface. To address this issue, the above-discussed examples may permit the freezing at multiple thickness by using various forms of such a lumen-based instrument. In one example, the outer lumen of the apparatus is inserted into the heart tissue and kept at a fixed position in the heart tissue and while the outer lumen is fixed, the inner lumen can be moved to the plurality of positions at varying depths in the heart tissue. Thus, at each depth, cryoablation is achieved. Because cryoablation is performed at the plurality of positions, the full thickness of the heart tissue is effectively treated, with the inner lumen staying inside outer lumen.

The liquid or gas for cryoablation is delivered to the delivery position by creating an opening in the inner lumen. The inner lumen either has an end-aperture or side aperture. The location of the aperture will determine the location of freezing. The inner lumen may be attached to (remotely-positioned) element that closes off the portion of the outer lumen to minimize the volume of the outer lumen. This may be implemented enhance the ability to achieve low temperatures and result in freezing. By limiting the location of delivery of freezing, an enhanced magnitude of freezing is achieved.

In another example such as illustrated by the end portion 145 of the inner lumen of FIG. 1C, the inner lumen delivers freeze energy by having the end portion 145 protrude outside the outer lumen 120. The outer lumen 120 is maintained in a stationary position relative to or outside of the target tissue site while the inner lumen is advanced into the tissue and with the end portion 145 moved to a plurality of delivery positions 140 to traverse the thickness of the heart tissue so that cryoablation is performed through the full thickness. A variety of mechanisms can be used to move the inner lumen including a plunger such as shown via 125 or 125′(e.g., in FIG. 1C with the outer dimensions remaining the same for the smaller protruding of the inner lumen).

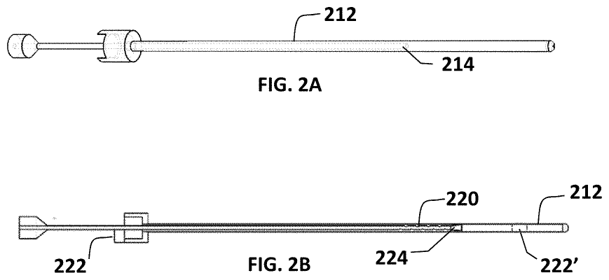

As illustrated in connection with FIGS. 2A and 2B, with the outer lumen 212 being moveable itself and/or relative to the inner lumen 214, delivery positions 220 of the inner lumen 214 may be in different forms, shapes and/or geometric locations along the length of the inner lumen for delivery at desired depths of the targeted tissue. For example, delivery positions 220 may be apertures or tapered (thickness) regions of the inner lumen wall. Also, such a lumen-based instrument may have a solid outer lumen and a center-perforated inner lumen (e.g., one or a few elongated delivery positions), in accordance with examples of the present disclosure.

More specifically, FIGS. 2A and 2B show examples of assembly types with each having an outer lumen 212 and an inner lumen 214, where the inner lumen 214 is allowed to slide within the boundaries of the outer lumen 212, and with the inner lumen 214 shown in a partially-extracted position relative the outer lumen 210. The inner lumen 214, in such examples, may have at least one end-located delivery position 224 (alone and/or in combination with other delivery positions 220) at or near the end of the inner lumen 214 to allow for flow of gas or liquids out of the inner lumen 212. In FIG.2B, such delivery positions 220 are depicted, for example, via perforations 220, running along the side or surface wall of the inner lumen 214 to allow for flow of gas or liquids out of the inner lumen 214 towards the wall and/or distal end of the outer lumen 212. Also shown is a blocker 222 (with a chamber from gas or liquid is released) which may be at or near the proximal end of the inner lumen or, as with blocker 222′, which may be at or near the distal end, and blocker 222′ may move with the distal end of the inner lumen.

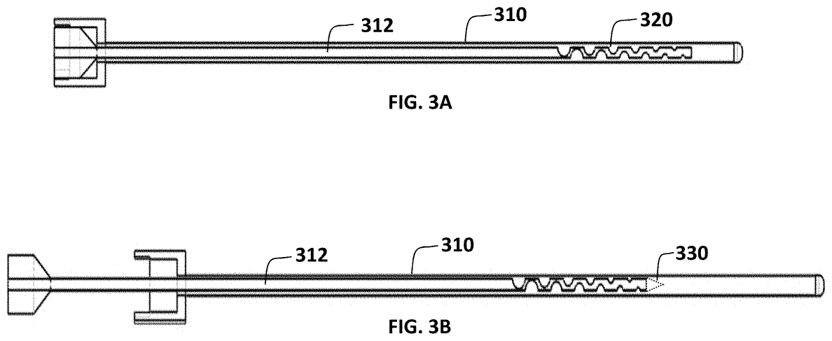

Also in accordance examples of the present disclosure, FIGS. 3A and 3B depict an apparatus or instrument with a solid outer lumen 310 and delivery positions 320 having a form integrated with a spirally-shaped or perforated inner lumen 312 in a fully-inserted position (FIG. 3A) and in a withdrawn position (FIG. 3B). Via the delivery positions 320 along the side wall(s) of the inner lumen 312 and with the inner lumen 312 being allowed to slide within the boundaries of the outer lumen 310, gas or liquids are permitted to flow out of the inner lumen 312 and into the outer lumen 310.

Further, FIG. 3B shows an optional pointed tip 330 which may be used to extend the inner lumen into the target tissue at depth(s). FIG. 3B shows a similar example with the inner lumen 312 repositions slightly, extending partially out of the outer lumen 310. This may be used as an example of repositioning to locate the inner lumen 312 to align to the selected depth(s) of the targeted tissue as targeted for the ablation treatment. Also shown is an, optional, conical or pointed tip 330 on the distal end of the inner lumen 312. This may be used when the outer lumen 310 has an open distal end and the inner lumen 312 is allowed to extend beyond the distal end of the outer lumen 310. In this case, the pointed end would allow ease of entry into surrounding tissue.

In yet further examples, aspects of the present disclosure are directed to sheath-based instruments used around an inner lumen which is in turn within an outer lumen. Such examples of sheath-based instruments may be used with the above-discussed aspects. In one such example, a sheath has at least a portion that is moveable relative to the inner lumen. By moving the sheath over the inner lumen which may have side apertures, it is possible to change the location of cryoablation. For example, by rotating, and/or advancing or withdrawing its relative position with respect to the positions along the length of the inner lumen), the sheath may be relocated to cover and uncover different side apertures to change the location of cryoablation, at varying depths.

In another such example, a sheath has at least a portion that is moveable relative to the inner lumen and is relatively gathered or reefed at the distal portion of the outer lumen of the apparatus. When the sheath is pulled or moved relative to an aperture in the inner lumen, the sheath is placed over the aperture will cause temperature of the liquid or gas to be applied. By moving or unreefing the sheath, a plurality of apertures at different positions will be exposed so that the location of cryoablation can be varied.

Such sheaths may not only be metal or plastic, but for reefing/unreefing sheaths the sheath materials may include or be composted of plastic, silicone, rubber, or another bendable or flexible metal.

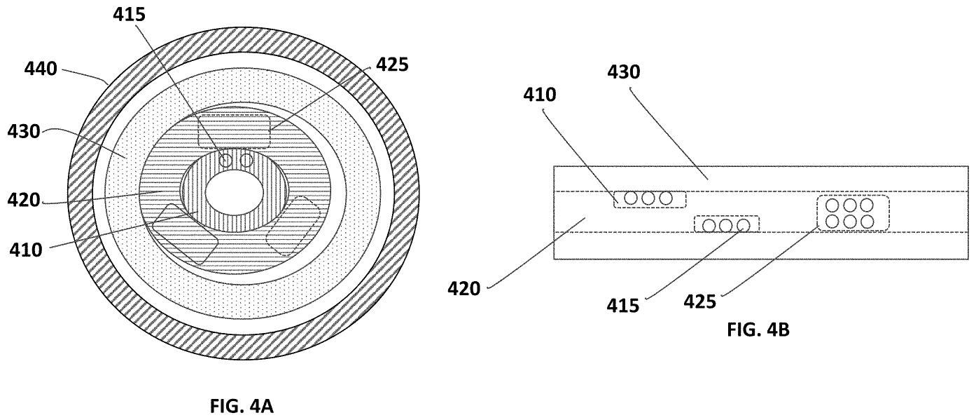

Turning now to FIGS. 4A and 4B, FIG. 4A shows a specific example of such a sheath-based assembly with FIG. 4A showing an interior, modified cross-sectional view of an apparatus or instrument with inner lumen 410, sheath/cover 420, and outer lumen 430, in accordance with examples of the present disclosure. FIG. 4B shows a perspective view of the apparatus or instrument of FIG. 4A with, for illustrative purposes, the outer lumen 430 having the sheath/cover 420 in dashed lines and having smaller portions of the inner lumen 410 within the sheath/cover 420.

In connection with both FIGS. 4A and 4B, the examples are used to show an inner lumen 410 for containing or presenting a gas or liquid (not shown) via perforations 415 (e.g., apertures partially or entirely through the wall thickness of the inner lumen 410), a sheath 420 with alignment locations 425 (e.g., perforations partially or entirely through the wall thickness) for passing otherwise-blocked energy from delivery perforations 415 of the inner lumen 410, an outer lumen 430 (e.g., as constructed as with any of the above embodiments), and a catheter 440. In one example for using the instrument of FIGS. 4A and 4B, the outer lumen is stable or secured (e.g., stickiness of the outer lumen to the tissue, and movement of the inner lumen or the sheath, relative to the position/movement of the sheath or the inner lumen, permits the delivery positions (perforations) 415 of the inner lumen to align with the alignment locations 425 of the sheath for delivery of the cryoenergy to the target tissue at the selected depth position(s). Such a sheath may also be constructed and used to curl back in response to certain temperature changes at tissue depth(s).

It may be appreciated that the sheath 420 is optional and, when used, may be inserted between the inner lumen 410 and the outer lumen 430 and this may occur entirely or partially before, during and/or after insertion of the inner lumen 410 and the outer lumen 430 into the catheter 440. Once between the inner lumen 410 and the outer lumen 430, the sheath 420 and/or the inner lumen 110 may be moved in various positions to align some or all of the delivery perforations 415 of the inner lumen 410 with the some or all of the alignment locations 425. By such movement, portions of the side perforations 220/320 may be blocked and aligned thereby directing the flow of gas or liquid. The sheath 420 may also be constructed to control amounts and/or positions, via such movement, for more controlled applications of the cryoenergy.

In another example, such a sheath 420 may be started in a reefed or gathered form near or around the delivery positions of the inner lumen and with movement of the inner lumen and/or sheath (similar to above) by withdrawing (unreeling) from, or further inserting (reefing) the sheath into, the outer lumen 430, one or more of the delivery positions of the inner lumen 410 are covered and/or covered, thereby allowing control over positioning and volumes of released gas or liquid out of the inner lumen 412 and/or selectively positioning and controlling the cooling rate via the position of the gathered sheath which may help to insulate the inner lumen (more so while gathered).

In yet other examples, such lumen-based assemblies may be implemented to include inner and outer lumens which permit for the inner lumen to change into a selectable (e.g., curved) shape when it protrudes from the end of the outer lumen. This may be realized, for example, by the temperature of the tissue reacting with the gas or liquid and/or the material (e.g., bimetal) in the inner lumen.

In still another example, the distal end of the outer lumen may include an inflatable balloon (e.g., as may be depicted at position 222′ of FIG. 2B) to assist in maintaining the stationary position of the assembly relative to the target tissue site. In yet another example, the distal end of the inner lumen may include an inflatable balloon (e.g., as may be depicted at position 224 of FIG. 2B) to assist in maintaining the stationary position when inserted into the target site.

As noted above, the various parts of the assemblies discussed herein can be made using various materials. The inner lumen and/or the outer lumen may be made, for example, from plastic or metal. The rotating sheaths may be made, for example, from metal or plastic. The reefing/unreeling sheaths may be made, for example, using plastic, silicone, rubber, or a bendable or flexible metal.

In more detailed/experimental embodiments consistent with various embodiments such as illustrated in connection with illustrated instruments, methods in accordance with the present disclosure involve performance of ex-vivo ablation on fresh porcine hearts. In connection with such detailed/experimental embodiments, the porcine hearts are submerged in circulated normal saline to ensure consistent tissue temperature during ablation. A needle-in-needle needle (OD:16 gauge) apparatus is connected to a liquid nitrogen reservoir (as in any of the figures herein), and the needle apparatus is inserted into the tissue (e.g., the myocardium from an epicardial approach). Two thermocouples are used to monitor changes in temperature within tissue and saline bath. One thermocouple is placed 2.5 mm away from the needle tip within the myocardium. Ice ball dimensions and time are studied are recorded. The results are significant with visualization of the formation of iceball after cryoablation in tissue, and a maximal cooling temperature of 41.0+0.2 C observed at 2.5 mm from needle trip in 7.5+0.5 minutes. The iceball is palpable at the end of the freeze, and remains palpable >120 seconds. Transmularity of lesions are achieved (e.g., 100%). Lesion width is at 1.1+0.5 cm. Accordingly, via such example instruments and/or methodology, needle cryoablation using liquid nitrogen is useful to create relatively large, transmural ventricular lesions.

Terms to exemplify orientation, such as upper/lower, left/right, top/bottom and above/below, may be used herein to refer to relative positions of elements as shown in the figures. It should be understood that the terminology is used for notational convenience only and that in actual use the disclosed structures may be oriented different from the orientation shown in the figures. Thus, the terms should not be construed in a limiting manner.

Based upon the above discussion and illustrations, those skilled in the art will readily recognize that various modifications and changes may be made to the various embodiments without strictly following the exemplary embodiments and applications illustrated and described herein. For example, methods as exemplified in the figures may involve steps carried out in various orders, with one or more aspects of the embodiments herein retained, or may involve fewer or more steps. Also, other technologies and related ranges may be used to extend/change example limits as disclosed herein as may be permitted by applicable technology. Such modifications do not depart from the true spirit and scope of various aspects of the disclosure, including aspects set forth in the claims.

What is claimed is:

1. An apparatus useful for a cryoablation procedure, the apparatus comprising:
an inner lumen, having a plurality of delivery positions along a length dimension of the inner lumen aligned with a depth dimension of tissue in a target tissue site, to use a gas or liquid to effect cryoablation at portions of the tissue at a plurality of depths within tissue;
an outer lumen to permit movement of the inner lumen relative to the outer lumen, and the outer lumen having an outer portion to facilitate movement of the inner lumen relative to the target tissue site; and
the inner lumen and the outer lumen being cooperatively configured wherein the movement of the inner lumen causes the temperature of the liquid or gas to be applied at different depth levels of the target tissue site via different ones of the plurality of delivery positions.

2. The apparatus of claim 1, wherein the inner lumen and the outer lumen are cooperatively configured to permit said at least one of a plurality of delivery positions of the inner lumen to move along the depth dimension into the target tissue site while maintaining the outer lumen in a stationary position relative to or outside of the target tissue site, and wherein the inner lumen is to use the gas or liquid to cause a decrease in temperature as it expands with a sudden drop in pressure or to use liquid at or below a temperature sufficiently cold to cryoablate portions of the tissue at a plurality of depths within tissue.

3. The apparatus of claim 1, wherein the inner lumen and the outer lumen are cooperatively configured to permit said at least one of a plurality of delivery positions of the inner lumen to move along the depth dimension into the target tissue site while maintaining the outer lumen in a stationary position within the target tissue site and said at least one of a plurality of delivery positions of the inner lumen within the outer lumen.

4. The apparatus of claim 1, further including a sheath having at least a portion of which is moveable relative to the inner lumen while covering said at least one of a plurality of delivery positions of the inner lumen, wherein movement of the sheath relative to the inner lumen causes the temperature of the liquid or gas to be applied.

5. The apparatus of claim 1, further including a sheath having at least a portion of which is moveable relative to the inner lumen while at least partially covering said at least one of a plurality of delivery positions of the inner lumen, wherein different ones of the plurality of delivery positions are geometrically arranged nonlinearly so that movement of the sheath relative to the inner lumen causes the temperature of the liquid or gas to be applied.

6. The apparatus of claim 1, further including a sheath having at least a portion of which is moveable relative to the inner lumen while at least partially covering said at least one of a plurality of delivery positions of the inner lumen, wherein each of the plurality of delivery positions is sized and geometrically arranged as a function of the different depth levels of the target tissue.

7. The apparatus of claim 1, further including a sheath having at least a portion of which is moveable from a first state being relatively gathered, reefed and/or unstressed, to a second state in which said at least one portion is less gathered, reefed and/or unstressed to expose said at least one of a plurality of delivery positions of the inner lumen and cause the temperature of the liquid to be applied.

8. The apparatus of claim 1, wherein an outer surface portion of the outer lumen is to be maintained or secured at a stationary position outside of the target tissue site with the outer lumen being further configured to release from the stationary position in response to a certain increase in temperature of the outer surface portion.

9. The apparatus of claim 1, further including a plunger to deliver pressure along the depth dimension and cause the movement of the inner lumen.

10. The apparatus of claim 1, further including a sheath to surround the inner lumen, and wherein the movement of the inner lumen is a rotation movement within the outer lumen to expose said at least one of a plurality of delivery positions of the inner lumen and to cause the temperature of the liquid to be applied.

11. The apparatus of claim 1, further including a catheter to guide the inner lumen and the outer lumen, and wherein the inner lumen defines said at least one of a plurality of delivery positions by an aperture.

12. The apparatus of claim 1, wherein the inner lumen defines said at least one of a plurality of delivery positions by a tapered layer portion of the inner lumen.

13. The apparatus of claim 1, wherein the inner lumen is coupled or connected to a more distal element that blocks flow of liquid or gas from traveling out of the inner lumen into the distal end of the outer lumen structure.

14. The apparatus of claim 1, further including a distal inflatable balloon and wherein the outer lumen is to use the distal inflatable balloon to maintain an outer surface portion of the outer lumen at a stationary position relative to or outside of the target tissue site.

15. The apparatus of claim 1, further including a distal inflatable balloon and wherein the inner lumen is to use inflatable balloon when inserted at depth into the tissue for delivery of gas or liquid towards or into achieve cryoablation of the surrounding tissue.

16. The apparatus of claim 1, wherein the inner lumen element can be pulled into curved shapes.

17. A method involving use of inner lumen within an outer lumen, the method comprising:
using the outer lumen to permit movement of the inner lumen relative to the outer lumen, and while the outer lumen has an outer portion applied relative to a target tissue site while the inner lumen moves within the outer lumen; and
with the inner lumen at least partially within the outer lumen and while a plurality of delivery positions along a length dimension of the inner lumen are aligned with a depth dimension of tissue in the target tissue site, using a temperature of gas or liquid at the plurality of delivery positions in the inner lumen to effect cryoablation of portions of the tissue at a plurality of depths within tissue, wherein the inner lumen and the outer lumen are cooperatively configured so that movement of the inner lumen causes the temperature of the liquid or gas to be applied at different depth levels of the target tissue site via different ones of the plurality of delivery positions.

18. The method of claim 17, wherein the inner lumen and the outer lumen are cooperatively configured to permit said at least one of a plurality of delivery positions of the inner lumen to move along the depth dimension into the target tissue site while maintaining the outer lumen in at a stationary position relative to or outside of the target tissue site, and wherein the inner lumen is to use the gas or liquid to cause a decrease in temperature as it expands with a sudden drop in pressure or to use liquid at or below a temperature sufficiently cold to cryoablate portions of the tissue at a plurality of depths within tissue.

19. The method of claim 17, wherein the inner lumen and the outer lumen are cooperatively configured to permit said at least one of a plurality of delivery positions of the inner lumen to move along the depth dimension into the target tissue site while maintaining the outer lumen in at a stationary position within the target tissue site and maintaining the inner lumen moveably within the outer lumen, and wherein the inner lumen is to use the gas or liquid to cause a decrease in temperature as it expands with a sudden drop in pressure or to use liquid at or below a temperature sufficiently cold to cryoablate portions of the tissue at a plurality of depths within tissue.

20. The method of claim 17, further including: a catheter to guide the inner lumen and the outer lumen; and a plunger to couple to the inner lumen, the outer lumen or both the inner lumen and outer lumen, and to cause the movement of the inner lumen.
